(19) United States
(12) Patent Application Publication
(10) Pub. No.: US 2021/0310949 A1
(43) Pub. Date: Oct. 7, 2021

(54) SYSTEM AND METHOD OF TRANSFORMING A PROTEIN TO EXHIBIT QUANTUM PROPERTIES AND APPLICATIONS THEREOF

(71) Applicant: INDIAN INSTITUTE OF SCIENCE EDUCATION AND RESEARCH, Punjab (IN)

(72) Inventors: Kamal Priya SINGH, Punjab (IN); Mehra Singh SIDHU, Punjab (IN); Biswajit PANDA, Punjab (IN)

(73) Assignee: INDIAN INSTITUTE OF SCIENCE EDUCATION AND RESEARCH, Punjab (IN)

(21) Appl. No.: 17/219,108

(22) Filed: Mar. 31, 2021

(30) Foreign Application Priority Data
Apr. 1, 2020 (IN) 202011004389

(51) Int. Cl. G01N 21/64; C09K 11/06; C07K 14/435

(52) U.S. Cl. CPC G01N 21/6402; C09K 11/06; C07K 14/43518; G01N 2201/0636; G01N 21/6486; G01N 2201/021; G01N 2201/06113; C07K 14/43536

(57) ABSTRACT

Disclosed herein is a novel phenomenon to create a nano-confined, dopant-free, electron spin-dependent fluorescence (SDF) in spider silk by fundamentally transforming its local molecular structure with femtosecond-pulses (206), having fluence below an ablation threshold. Electron-spin dependence of the fluorescent patterns created on the silk sample are confirmed by measuring the fluorescence intensity at different microwave frequencies. The fluorescent intensity exhibits microwave magnetic resonances at 2.88 GHz and 1.44 GHz at room-temperature. The SDF in laser-transformed silk can thereby enable a new-class of tough yet elastic silk-based quantum sensor and hybrid nano-mechanical ultrasensitive cantilevers on a micro-chip. X-ray diffraction (XRD), Raman-spectroscopy, direct atomistic imaging with high-resolution transmission electron microscopy (HR-TEM) and model-building studies are carried out to exhibit the change in the molecular structure and unveil creation of crown-ring like structure in nanocrystals of fluorescent silk with localized electrons possessing mid-gap states.

### CROSS-REFERENCE TO RELATED APPLICATIONS

This application claims priority to Indian Application No. 202011004389, filed on Apr. 1, 2020, the contents of which is herein incorporated by reference in its entirety.

### FIELD OF INVENTION

The present disclosure relates to a system and a method for inducing structural modifications in protein-based biomaterials and more particularly, the present disclosure relates to creating and detecting electron-spin dependent quantum properties in the protein-based biomaterials at room temperature by modifying its molecular structure using laser and without causing any damage to the protein structure.

### BACKGROUND OF INVENTION

Studying the transformation of proteins/synthetic proteins or protein-based biomaterials is of fundamental interest for wide range of fields including, physics, chemistry, material processing, precision technologies to biological processes, etc. In particular, the proteins or protein-based biomaterials have been of great interest to understand and mimic their unmatched conformational changing property under natural or unnatural conditions. Intriguingly, the building-blocks of proteins/protein-based biomaterials/fibers can rearrange themselves in enormous and diverse combinations that remain unearthed so far and can be explored in lab using a continuous-wave or ultra-fast laser perturbation. For example, it is unknown whether the structure of protein, for example, in silk, can be locally transformed to create electron spin-dependent fluorescence at room temperature. Observation of such quantum phenomenon in proteins, protein-based biomaterials like silk is unexpected and challenging.

Various research reports have been published so far where, the silk or other proteins materials/fibers was infiltrated with various nano-dopants to impart fluorescence, enhance its toughness and modulate its optical, electrical and magnetic properties [Iizuka, T. et al. Advanced Functional Materials. (2013), Samal, S. K. et al., ACS Appl. Mater. Interfaces (2015)]. Previously, Lee, et al. (2009), Babb, et al. (2019), have used femtosecond (fs) pulses of light for nano-processing materials and inducing transient or permanent modification in materials such as change in refractive index of dielectrics, multi-petahertz control of light-induced electric conductivity in amorphous SiO₂, synthesis of PbTiO₃ supercrystals with 3D nanoscale periodicity (Steven E et al. (2013) and Flores, M et al. (2008)), fabrication of black-silicon by sulphur hyperdoping for enhanced infrared absorption, and writing of color centers in diamond and SiC for quantum applications (Chen Y et al. (2017 and 2019)). However, whether a protein can be transformed to create electron-spin dependent quantum effects at room temperature remains unknown due to various challenges. For instance, the creation of electron-spin-dependent quantum phenomenon in silk at room-temperature is challenging due to two fundamental reasons. First, the thermal energy at T=300K (kT≈25 meV) is about 1000 times higher than typical energy-scale corresponding to spin-levels (ΔE≈10 μeV). Second, the pristine silk fiber is a few micron in diameter which is too large to show any nanoscale quantum confinement effects. In addition, high optical transparency of silk precludes optical detection of such quantum effects.

Thus, there is a need for a method that facilitates creation of electron-spin-dependent quantum phenomenon in a protein by overcoming the various aforementioned challenges and without destroying the molecular structure of the protein. Further, there is also a need for exploring the suitability of the created quantum phenomenon in proteins/synthetic proteins or protein-based biomaterials for a wide range of applications, for example, in the broadband quantum sensing such as acoustic, magnetic, mechanical and temperature sensing.

### OBJECTIVES OF INVENTION

An objective of the present invention is to create a nano-confined, dopant-free, spin-dependent visible fluorescence (SDF) in protein-based biomaterials.

Another objective of the present invention is to provide a method for transforming local structure of protein-based biomaterials to a new form without destroying the molecular structure.

Yet another objective of the present invention is to provide a system and method for creating electron spin dependent fluorescence at nano scale level in a protein.

Yet another objective of the present invention is to provide a system and method for creation of electron spin dependent bulk fluorescence in a protein

Yet another objective of the present invention is to provide systems and methods employing transformed proteins of the present invention for applications in various fields.

### SUMMARY OF INVENTION

The present disclosure overcomes one or more shortcomings of the prior art and provides additional advantages discussed throughout the present disclosure. Additional features and advantages are realized through the techniques of the present disclosure. Other embodiments and aspects of the disclosure are described in detail herein and are considered a part of the claimed disclosure.

In one non-limiting embodiment of the present disclosure, a system for creating electron spin dependent fluorescence at nano scale in a protein is disclosed. The system comprises a translation stage with the protein placed thereupon. The translation stage is capable of moving along at least one of X, Y and Z direction. The system further comprises a femtosecond pulse laser placed diagonally to the translation stage and configured to generate a femtosecond pulse to create a fluorescence pattern at nanoscale level on the protein sample by moving the translation stage along the at least one of X, Y and Z direction. The generated femtosecond pulse is directed towards the protein sample by a first focusing mirror placed between the femtosecond pulse laser and the translation stage in such a manner that the femtosecond laser is in a vertical direction with respect to the first focusing mirror and the translation stage in a horizontal direction with respect to the first focusing mirror. The fluorescence pattern at nanoscale is created by irradiating the protein with said femtosecond pulse at one or more irradiation fluences in a room temperature. The system further comprises a continuous wave laser placed diagonally to the translation stage and configured to generate a laser beam to detect the created fluorescence pattern on the protein sample within a wavelength range of visible light. The generated laser beam is directed towards the protein sample by a second focusing mirror placed between the continuous wave laser and the translation stage in such a manner that the continuous wave laser is in a vertical direction with respect to the second focusing mirror and the translation stage in a horizontal direction with respect to the second focusing mirror. The system further comprises a microwave resonator capable of providing one or more frequencies and placed in proximity to the protein on which the fluorescence pattern is created. Further, the system comprises a detector placed axially from the translation stage, configured to detect a magnetic resonance of the created fluorescence pattern at least one of the one or more frequencies.

In one non-limiting embodiment of the present disclosure, a system for creating electron spin dependent bulk fluorescence in a protein is disclosed. The system comprises a translation stage with the protein placed thereupon, where the translation stage is capable of moving along at least one of X, Y and Z direction. The system further comprises a continuous wave laser placed diagonally to the translation stage and configured to generate a laser beam to create and detect the created fluorescence pattern on the protein sample within a wavelength range of visible light, The generated laser beam is directed towards the protein sample by a focusing mirror placed between the continuous wave laser and the translation stage in such a manner that the continuous wave laser is in a vertical direction with respect to the focusing mirror and the translation stage in a horizontal direction with respect to the focusing mirror and the fluorescence pattern is created and detected by irradiating the protein using the laser beam at room temperature and at predetermined pressure. The system further comprises a microwave resonator capable of providing one or more frequencies and placed in proximity to the protein on which the fluorescence pattern is created. The system further comprises a detector placed axially from the translation stage, configured to detect a magnetic resonance of the created fluorescence pattern at least one of the one or more frequencies.

In one non-limiting embodiment of the present disclosure, a method of creation of electron spin dependent fluorescence at nano scale level in a protein is disclosed. The method comprises creating a fluorescence pattern at the nanoscale level on the protein by irradiating the protein using femtosecond pulses generated from a femtosecond pulse laser at one or more irradiation fluences at room temperature. A value of the one or more irradiation fluences is less than a value of an ablation threshold of the protein. The method further comprises detecting the created fluorescence pattern by directing a laser beam towards the transformed protein. The laser beam is generated by a continuous wave laser having a wavelength within a wavelength range of visible light. The method further comprises providing a microwave resonator, placed in proximity to the protein on which fluorescence pattern is created, wherein the microwave resonator is capable of providing a plurality of frequencies. Further, the method comprises confirming an electron spin dependent fluorescence pattern by detecting, at a detector, a magnetic resonance of the created fluorescence pattern for at least one of the plurality frequencies.

In one non-limiting embodiment of the present disclosure, a method of creation of electron spin dependent bulk fluorescence in a protein is disclosed. The method comprises creating and detecting a bulk fluorescence pattern on the protein by irradiating the protein using a laser beam at room temperature and at predetermined pressure. The laser beam is generated by a continuous wave laser having a wavelength within a wavelength range of visible light. The method further comprising providing a microwave resonator, placed in proximity to the protein on which fluorescence pattern is created, wherein the microwave resonator is capable of providing a plurality of frequencies. The method further comprises confirming an electron spin dependent fluorescence pattern by detecting, at a detector, a magnetic resonance of the created fluorescence pattern for at least one of the plurality of frequencies.

In one non-limiting embodiment of the present disclosure, a transformed protein having altered molecular structure and without causing any damage to the protein structure, obtained by the method or the system as described in the non-limiting embodiments described above is disclosed. The protein exhibits modified quantum properties and fluorescence that is nano-confined, dopant-free and electron spin-dependent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts absorption spectra of a protein-based biomaterial (e.g., pristine silk sample) in accordance with an embodiment of the present disclosure;

FIG. 2A depicts an experimental setup 200 for creation and detection of nanoscale fluorescent pattern on the pristine silk sample at room temperature in accordance with an embodiment of the present disclosure;

FIG. 2B depicts an experimental setup 200A for creation and detection of bulk fluorescent pattern on the pristine silk sample at room temperature in accordance with an embodiment of the present disclosure;

FIG. 3 depicts a method 300 for creation and detection of fluorescent pattern on the pristine silk sample at room temperature in accordance with an embodiment of the present disclosure;

FIGS. 4A and 4B depict the fluorescence images obtained using electron multiplying charge-couple device (EMCCD) in accordance with an embodiment of the present disclosure;

FIGS. 4C and 4D depict the bright field microscopy images of the transformed silk sample in accordance with an embodiment of the present disclosure.

FIG. 5 depicts a fluorescent intensity versus fluence plot for the created fluorescent pattern in accordance with an embodiment of the present disclosure;

FIG. 6A depicts the fluorescence spectra at two excitation powers of a continuous wave laser in accordance with an embodiment of the present disclosure;

FIG. 6B depicts time-correlated single-photon counting measurement of fluorescence life-time in accordance with an embodiment of the present disclosure;

FIG. 7 depicts an experimental setup 700 for optical detection of magnetic resonance in silk sample in accordance with an embodiment of the present disclosure;

FIG. 8 depicts a method 800 for optical detection of magnetic resonance in silk sample in accordance with an embodiment of the present disclosure;

FIG. 9 depicts a plot of fluorescence intensity versus microwave frequency at room temperature in accordance with an embodiment of the present disclosure;

FIG. 10 depicts the XRD patterns of laser treated silk (or transformed silk) and native (or pristine) silk in accordance with an embodiment of the present disclosure;

FIG. 11 depicts the Raman spectra of laser treated silk (or transformed silk) and native (or pristine) silk in accordance with an embodiment of the present disclosure;

FIG. 12 depicts the transformation of hydrogen-bonded β-strands of pristine silk into C—O—C bonded structure of laser treated silk (or transformed silk) in accordance with an embodiment of the present disclosure;

FIG. 13 depicts the HR-TEM image of the laser treated silk (or transformed silk) in accordance with an embodiment of the present disclosure;

FIG. 14 depicts a comparison of lattice periodicity of nano-crystals in fluorescent (or transformed or laser treated) and native silk from XRD and HR-TEM in accordance with an embodiment of the present disclosure;

FIG. 15 depicts enthalpy of formation for native and C—O—C bonded β-strands in accordance with an embodiment of the present disclosure;

FIG. 16 depicts an experimental energy level diagram of spin-dependent fluorescent (SDF) silk in accordance with an embodiment of the present disclosure;

FIG. 17 depicts an experimental setup 1700 of fluorescent silk-based quantum thermometer in accordance with an embodiment of the present disclosure;

FIG. 18 depicts a method 1800 for fluorescent silk-based quantum thermometer in accordance with an embodiment of the present disclosure;

FIG. 19A depicts a shift in magnetic resonance induced by laser heating in accordance with an embodiment of the present disclosure;

FIG. 19B depicts a shift in frequency as a function of temperature in accordance with an embodiment of the present disclosure;

FIG. 20 depicts a micro-chip 2000 with nano-mechanical silk-cantilever (device 1) and string-resonators (device-2) in accordance with an embodiment of the present disclosure;

FIG. 21 depicts a method 2100 for employing transformed silk as nano-mechanical silk-cantilever (device 1) and string-resonators (device-2) in accordance with an embodiment of the present disclosure; and

FIG. 22 depicts optical detection of mechanical response by acoustic excitation in accordance with an embodiment of the present disclosure;

The figures depict embodiments of the disclosure for purposes of illustration only. Further, the experimental results and/or data about the spider silk (or simply silk) mentioned/depicted in the figures and in the present disclosure are merely for purpose of illustration only and thus, various set-ups, embodiments and its implementation details described in this disclosure is limited not only to such silk but also may be applied to various other proteins or protein-based bio-materials. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. It should be appreciated by those skilled in the art that the conception and specific embodiment disclosed may be readily utilized as a basis for modifying or designing other structures for carrying out the same purposes of the present disclosure.

The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

Disclosed herein is a novel phenomenon to create a nano-confined, dopant-free, electron spin-dependent fluorescence (SDF) in protein based biomaterial e.g, spider Araneus neoscona silk and Bombyx mori silkworm silk by fundamentally transforming its local molecular structure with femtosecond (fs)-pulses, having fluence below an ablation threshold. Electron-spin dependence of the fluorescent patterns created on the silk sample are confirmed by measuring the fluorescence intensity at different microwave frequencies. The fluorescent intensity exhibits microwave magnetic resonances at 2.88 GHz and 1.44 GHz at room-temperature. The SDF in laser-transformed silk can thereby enable a new-class of tough yet elastic silk-based quantum sensor and hybrid nano-mechanical ultrasensitive cantilevers on a micro-chip. X-ray diffraction (XRD), Raman-spectroscopy, direct atomistic imaging with high-resolution transmission electron microscopy (HR-TEM) and model-building studies are carried out to exhibit the change in the molecular structure and unveil creation of crown-ring like structure in nanocrystals of fluorescent silk with localized electrons possessing mid-gap states.

The present disclosure further describes a novel phenomenon for creating a bulk fluorescent pattern in protein-based biomaterial e.g, spider dragline silk by fundamentally transforming its local molecular structure by either laser beam irradiation, high pressure or simple heating in air, vacuum or Argon environment.

Although, the present disclosure and the figures mentioned therein depicts various electrical, magnetic and optical property of spider-silk (or simply silk) sample, a person skilled in the art may note that the same is merely for the purpose of illustration only and nowhere limits the disclosure only to this silk. Instead, the embodiments of the present disclosure may be applied to various other proteins or protein-based bio-materials including but not limited to spider silk.

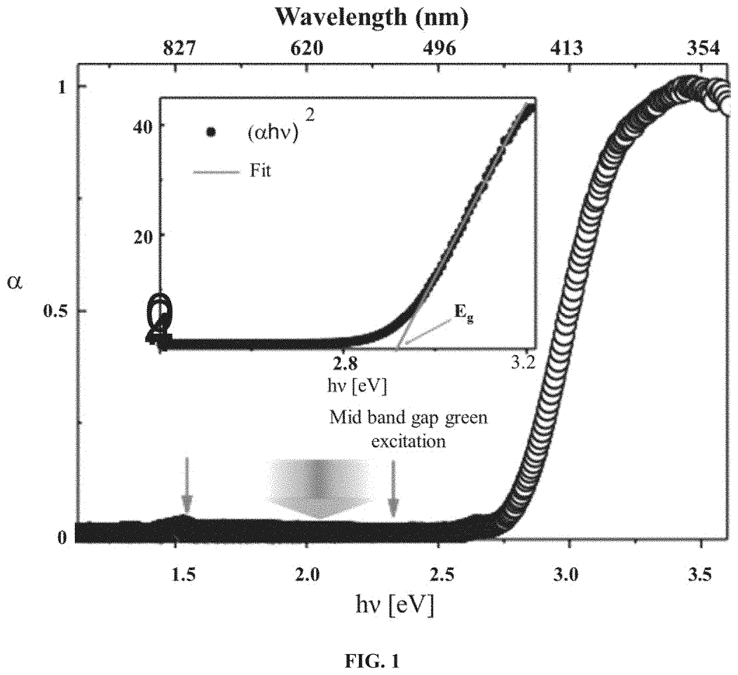

As briefly discussed in the background section of the disclosure, pristine silk (interchangeably referred to as “native silk”) does not exhibit fluorescence in the visible range of spectrum due to its high optical transparency, thermal energy and large diameter. To confirm this, optical studies are carried out on pristine silk and the results are illustrated in FIG. 1 which shows an absorption spectra of pristine silk. As illustrated in FIG. 1, pristine silk is highly transparent from infrared to visible spectral range as absorption of light in the wavelength range of around 450-850 nm is almost zero. Further, the inset of FIG. 1 shows a Tauc plot ((αhv)² vs photon energy, hv, where a is the absorbance) using which the bandgap, E_{g} of pristine silk is calculated to be 2.9±0.1 eV which is comparable to the known values of the bandgaps in amorphous materials and other proteins, thereby confirming that pristine silk does not show any fluorescence.

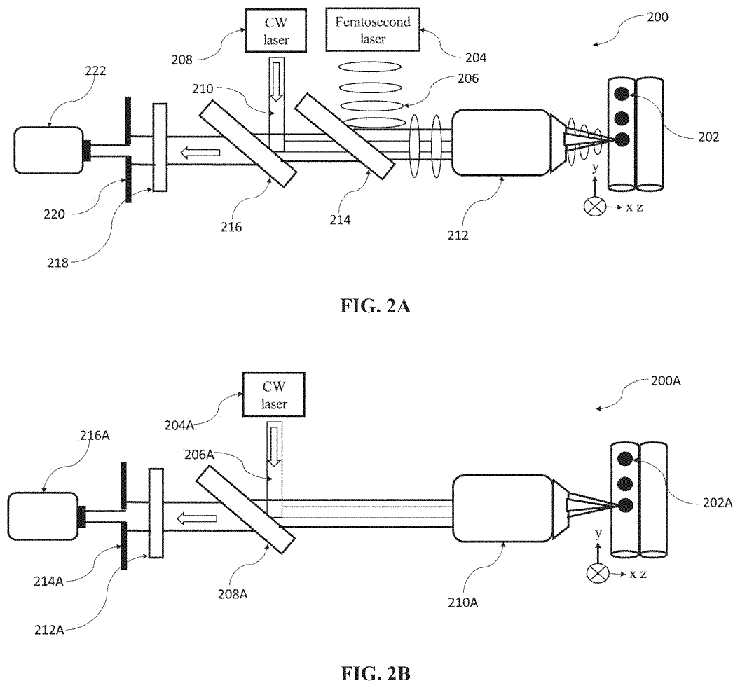

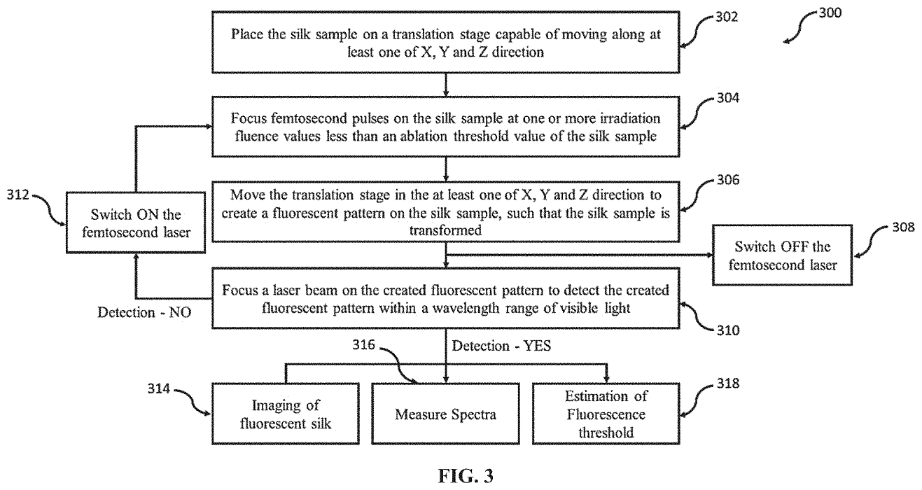

FIG. 2A depicts an experimental setup 200 (herein after referred to as “setup 200”) for creation and detection of nanoscale fluorescent pattern on the pristine silk sample in accordance with an embodiment of the present disclosure. The setup 200 comprises a translation stage (not shown) capable of moving along the X, Y and Z directions. The pristine silk sample is placed upon the translation stage. The setup 200 further comprises a femtosecond pulse laser 204 placed diagonally to the translation stage and configured to generate femtosecond pulses 206. The femtosecond pulses 206 are directed towards the pristine silk sample by means of a first focusing mirror 214 placed in the path of the femtosecond pulses 206 to transform the pristine silk sample (interchangeably referred as “transformed silk” or “laser treated silk”) in order to create a fluorescent pattern 202. The setup 200 further comprises a continuous wave (CW) laser 208 configured to generate a CW laser beam 210 at a particular excitation wavelength within the visible range of the electromagnetic spectrum. The CW laser beam 210 is directed towards the transformed silk sample by means of a second focusing mirror 216 so as to detect the created fluorescent pattern 202 on the transformed silk sample. The setup 200 further comprises a filter 218 to avoid excitation wavelength in fluorescence detection on the transformed silk sample. The setup further comprises a pin-hole 220 and a detector 222 to obtain fluorescence images of the transformed silk sample. In one embodiment, the first focusing mirror 214 and the second focusing mirror may be a dichroic mirror. In one embodiment, the filter 218 may be a notch filter. In one embodiment, the detector 222 may be an electron multiplying charge coupled device (EMCCD). However, it may be understood may a skilled person that different combinations of the focusing mirrors 214, 216, filter 218 and detector 222 may be used.

FIG. 3 depicts a method 300 for creation and detection of nanoscale fluorescent pattern on the pristine silk sample in accordance with an embodiment of the present disclosure.

The order in which the method 300 is described is not intended to be construed as a limitation, and any number of the described method blocks can be combined in any order to implement the method. Additionally, individual blocks may be deleted from the methods without departing from the spirit and scope of the subject matter described herein.

At block 302, the method 300 may include placing the pristine silk sample on the translation stage that is capable of moving in the X, Y and Z direction in order to create a desired fluorescent pattern 202 on the pristine silk sample with a precision of at least 100 nm.

At block 304, the method 300 may include focusing femtosecond pulses 206 generated from the femtosecond laser 204 on the pristine silk sample by means of the first focusing mirror 214 placed in between the femtosecond laser 204 and the translation stage on which the pristine silk sample is placed. The pristine silk sample is irradiated with a single pulse fluence, F, ranging between 1-1000 mJ/cm². The single pulse fluence, F is chosen in such a manner that the fluences lie below an ablation threshold of the pristine silk sample to prevent the molecular structure of the pristine silk sample from being destroyed.

It may, be noted that the range of fluence, F, described herein is chosen for a silk sample. However, the range of fluence may differ if a different protein-based biomaterial is used.

In one embodiment, the femtosecond laser 204 has a wavelength range of 700-800 nm, energy range of 2 nJ-2 mJ and the pulse width of the generated femtosecond pulses lies 206 within a range of 7-25 femtoseconds.

In one embodiment, the first focusing mirror 214 is placed in between the femtosecond laser 204 and the translation stage in such a manner that the femtosecond laser 204 is in a vertical direction with respect to the first focusing mirror 214 and the translation stage in a horizontal direction with respect to the first focusing mirror 214.

It may however be understood by a skilled person that the femtosecond laser 204, the first focusing mirror 214 and the translation stage may be arranged in any other suitable manner such that the femtosecond pulses 206 are directed towards the pristine silk sample.

At block 306, the method 300 may include moving the translation stage in at least one of X, Y and Z directions to transform the pristine silk sample in order create the fluorescent pattern 202 on the pristine silk sample. In one embodiment, the pristine silk sample is raster scanned by the femtosecond pulses 206 by moving the translation stage in at least one of X, Y and Z directions in order to create the fluorescent pattern.

In one embodiment, the created fluorescent pattern 202 may be a fluorescent nano-dot or a fluorescent line or any other fluorescent pattern.

At block 308, the method 300 may include switching OFF the femtosecond laser 204 in order to detect the created fluorescent pattern 202 within the visible range of electromagnetic spectrum by employing a continuous wave (CW) laser 208 having an excitation wavelength within the visible range of electromagnetic spectrum.

At block 310, the method 300 may include focusing a CW laser beam 210 at one or more excitation powers (≤1 mW) generated from the CW laser 210 on the transformed silk sample to detect the created fluorescent pattern 202 by means of the second focusing mirror 216 placed in between the CW laser 208 and the translation stage on which the transformed silk sample is placed.

In one embodiment, the second focusing mirror 216 is placed in between the CW laser 208 and the translation stage in such a manner that the CW laser 208 is in a vertical direction with respect to the second focusing mirror 216 and the translation stage is in a horizontal direction with respect to the second focusing mirror 216.

In one embodiment, the CW wave laser 208 may have a wavelength of 532 nm corresponding to green excitation in order to detect the created fluorescent pattern 202 with the visible range of electromagnetic spectrum.

If a visible fluorescent pattern is observed by the detector 222, the method 300 proceeds to blocks 314-318. However, if a visible fluorescent pattern is not detected, the method 300 proceeds to block 312.

At block 312, the method 300 may include switching ON the femtosecond laser 202 in order to irradiate the pristine silk sample with femtosecond pulses 206 with another fluence, F, different from the one chosen at block 304. The method 300, then follows the sequence of steps following block 304.

However, if a visible fluorescent pattern is detected at block 310, the method 300 further proceeds to blocks 314-318 where the steps of imaging of fluorescent silk, measuring the fluorescent spectra and estimation of fluorescence threshold is performed.

In the embodiment, depicted in FIGS. 2A and 3, the molecular structure of pristine silk is transformed by subjecting pristine silk to femtosecond laser pulses 206. However, it must be noted that the molecular structure of pristine silk can be transformed by subjecting pristine silk to high pressure in the range 0.1-10 GPa, or by simply heating pristine silk in air, vacuum or argon environment.

Further, in one embodiment, the setup 200 depicted in FIG. 2A is modified to create a bulk fluorescence pattern on the pristine silk. In accordance with the embodiment to create and detect bulk fluorescent pattern. The setup 200A comprises a translation stage (not shown) capable of moving along the X, Y and Z directions. The pristine silk sample is placed upon the translation stage. The setup 200A further comprises a continuous wave laser 204A placed diagonally to the translation stage and configured to generate a CW laser beam 206A at a particular excitation wavelength within the visible range of the electromagnetic spectrum. The CW laser beam 206A is directed towards the pristine silk sample by means of a focusing mirror 208A so as to create and detect the bulk fluorescent pattern 202A on the transformed silk sample. The setup 200A further comprises a filter 212A to avoid excitation wavelength in fluorescence detection on the transformed silk sample. The setup further comprises a pin-hole 214A and a detector 216A to obtain fluorescence images of the transformed silk sample. In one embodiment, the focusing mirror 208A may be a dichroic mirror. In one embodiment, the filter 212A may be a notch filter. In one embodiment, the detector 216A may be an electron multiplying charge coupled device (EMCCD). However, it may be understood may a skilled person that different combinations of the focusing mirror 208A, filter 212A and detector 216A may be used.

In one embodiment, the pristine silk sample is raster scanned by the laser beam 206A by moving the translation stage in at least one of X, Y and Z directions in order to create the bulk fluorescent pattern 202A. Further, in one embodiment, the bulk fluorescent pattern 202A comprises at least one multiple dots, fluorescent line or any other fluorescent pattern.

Further, in one embodiment, the bulk fluorescent pattern 202A may be created by subjecting pristine silk to high pressure within a range 0.1-10 GPa, or by simply heating pristine silk in air, vacuum or argon environment.

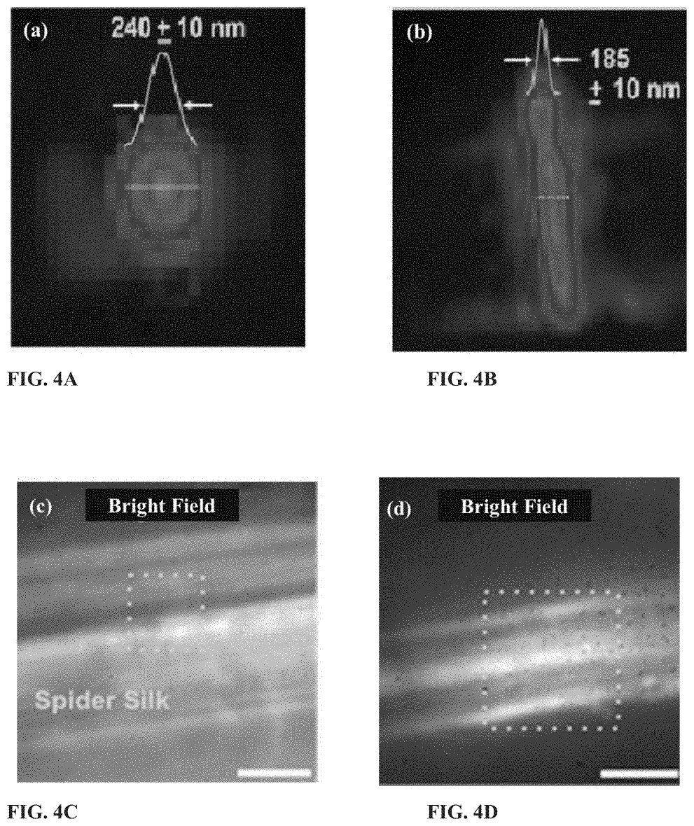

FIGS. 4A and 4B depict the fluorescence images obtained using electron multiplying charge-couple device (EMCCD) performed at block 314 of the method 300 in accordance with an embodiment of the present disclosure. FIG. 4A depicts the created fluorescent pattern 202 in the form of a nano-dot. The full-width half-maxima of the created nano-dot is found to be around 240±10 nm. Further, FIG. 4B depicts the fluorescent pattern 202 in the form of a line obtained by raster scanning the pristine silk sample at one or more single-pulse fluences, F, obtained from the femtosecond laser 202. The full-width half-maxima of the created line is found to be around 185±10 nm with a length of around 4 μm. However, in one embodiment, the full-width half maxima of the created fluorescent pattern may lie within a range of 100-250 nm depending upon the type of the fluorescent pattern.

FIGS. 4C and 4D depict the bright field microscopy images of the transformed silk sample in accordance with an embodiment of the present disclosure. The bright field microscopy images depict that no visible damage is observed on the transformed silk sample.

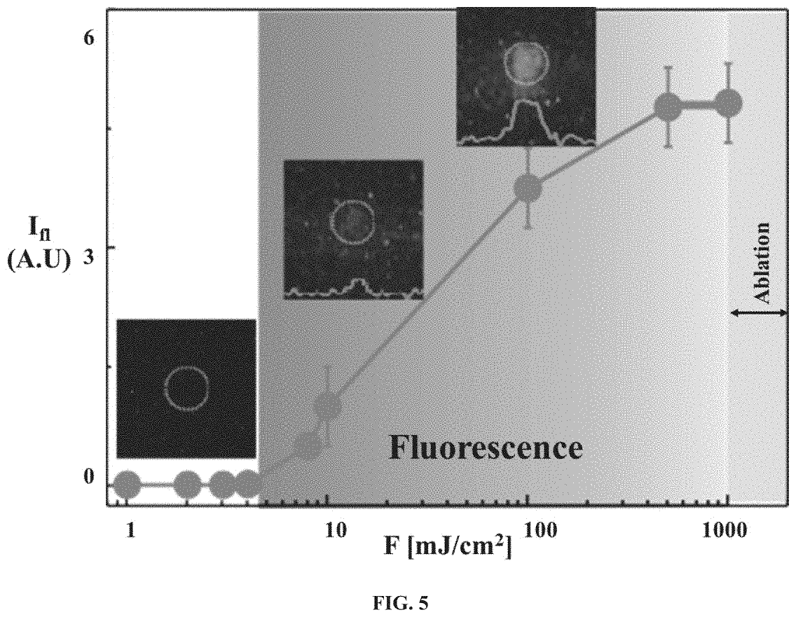

FIG. 5 depicts experimental data showing a graph of a fluorescence intensity (I_{η}) versus fluence (F) plot for the created fluorescent pattern in the silk. From the same, one may estimate the fluorescence threshold in accordance with block 318 of the method 300 illustrated and described above in the present disclosure. As seen from FIG. 5, the threshold fluence, Fₜₕ, is observed to be 8±1 mJ/cm² as below Fₜₕ, no visible fluorescence is observed. Further, FIG. 5 depicts an increase in the fluorescence intensity (I_{η}) with fluence and saturated below the ablation threshold of 1000 mJ/cm². Thus, from this experimental data it may be concluded that there exists a wide range of fluence, F, where the creation of fluorescence in silk sample is damage free.

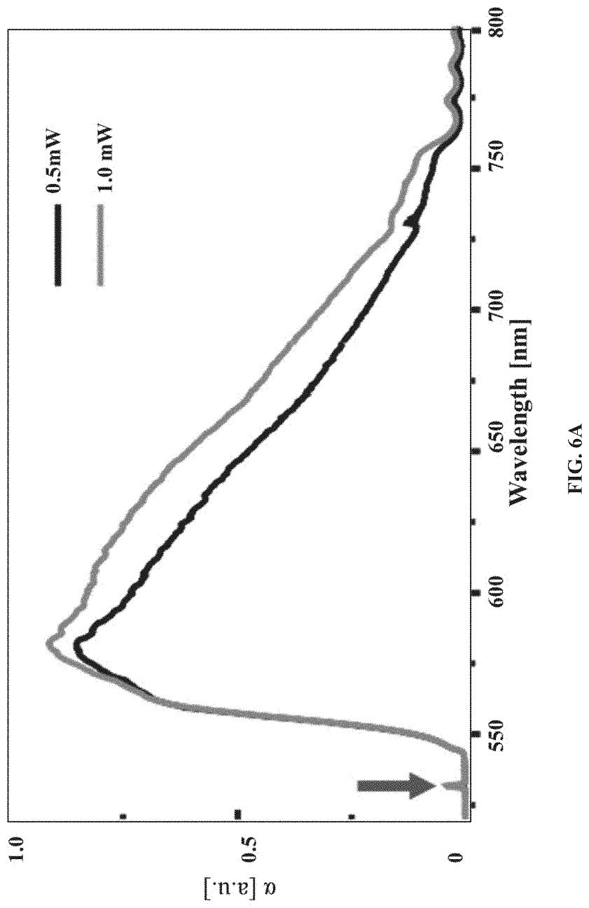

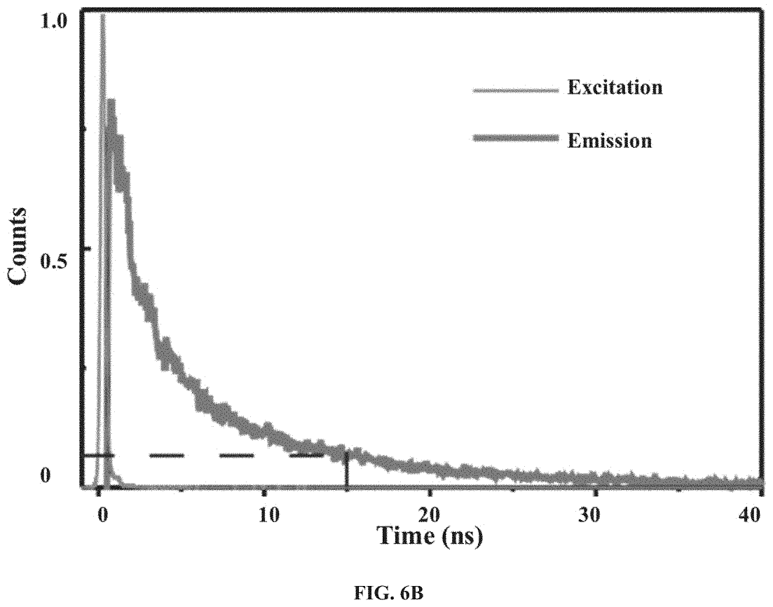

FIG. 6A depicts the fluorescence spectra at two excitation powers of a continuous wave laser 208 in accordance with an embodiment of the present disclosure. The fluorescence spectra for the transformed silk sample as depicted in FIG. 6A are broadband with a sharp cut-off near 550 nm, a maximum around 580 nm and a slowly falling tail extending up to 750 nm. The central wavelength is observed to be near 630 nm with a FWHM of 100 nm.

FIG. 6B depicts time-correlated single-photon counting measurement of fluorescence life-time in accordance with an embodiment of the present disclosure. The time-corelated single photon counts (TCSPC) is performed using picosecond laser excitation at a wavelength of 514 nm. The fluorescence life-time for the transformed silk sample is observed to be within a range of 10-50 ns which confirms optical emission from the laser-treated silk as fluorescence.

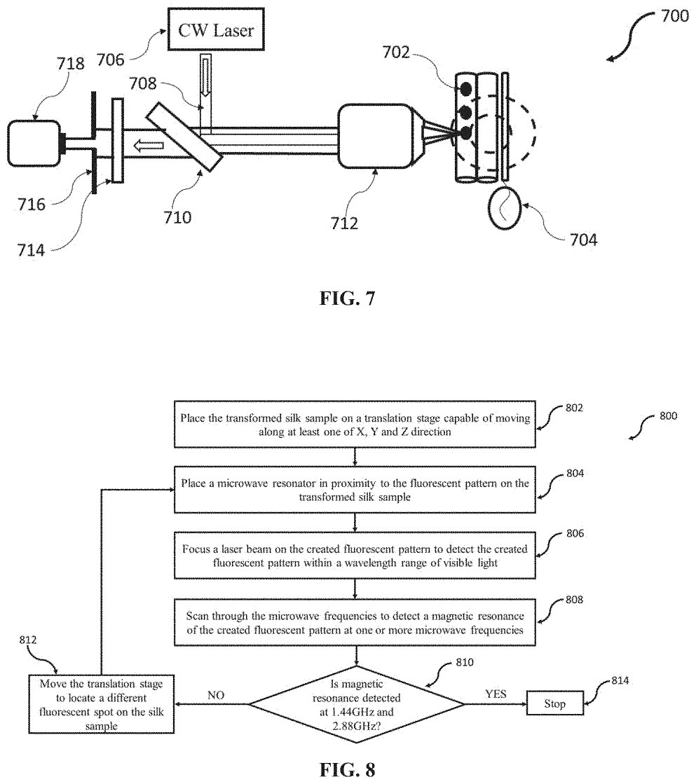

FIG. 7 depicts an experimental setup 700 (herein after referred to as “setup 700”) for optical detection of magnetic resonance in silk sample to confirm electron-spin dependence of the created fluorescent pattern 702 in accordance with an embodiment of the present disclosure. The setup 700 comprises a translation stage capable of moving along the X, Y and Z directions. The transformed silk sample is placed upon the translation stage. The setup 700 further comprises a continuous wave (CW) laser 706 placed diagonally to the translation stage configured to generate a CW laser beam 708 at a particular excitation wavelength within the visible range of the electromagnetic spectrum. The CW laser beam 708 is directed towards the transformed silk sample by means of a focusing mirror 710 so as to detect the created fluorescent pattern 702 on the transformed silk sample. The setup 700 further comprises a microwave resonator 704 placed in close proximity to the transformed silk sample and capable of generating microwave frequencies within a range 1.4-3.0 GHz. The setup 700 further comprises a filter 714 to avoid excitation wavelength in fluorescence detection on the transformed silk sample. The setup further comprises a pin-hole 716 and a detector 718 to detect a change in the fluorescence intensity with microwave frequencies in order to detect magnetic resonance at one or more microwave frequencies. In one embodiment, the focusing mirror 710 and may be a dichroic mirror. In one embodiment, the filter 714 may be a notch filter. In one embodiment, the detector 718 may be an avalanche photo diode (APD) or a single photon detector. However, it may be understood may a skilled person that different combinations of the focusing mirror 710, filter 714 and detector 718 may be used. Further, in one embodiment, the microwave resonator 704 is a broadband wire resonator having a diameter of 20 μm and is fed with a current source of −10 dBm. However, it may be understood that different types of microwave resonators may be used.

FIG. 8 depicts a method 800 for optical detection of magnetic resonance in silk sample in accordance with an embodiment of the present disclosure.

The order in which the method 800 is described is not intended to be construed as a limitation, and any number of the described method blocks can be combined in any order to implement the method. Additionally, individual blocks may be deleted from the methods without departing from the spirit and scope of the subject matter described herein.

At block 802, the method 800 may include placing the transformed silk sample on the translation stage that is capable of moving in the X, Y and Z direction in order to detect the created fluorescent pattern 702 on the transformed silk sample.

At block 804, the method 800 may include placing a microwave resonator 704 in close proximity to the transformed silk sample and capable of generating microwave frequencies within a range 1.4-3.0 GHz. In one embodiment, the distance at which the microwave resonator 704 is placed with respect to the transformed silk sample is 100-200 nm.

At block 806, the method 800 may include focusing a CW laser beam 708 at one or more excitation powers (≤1 mW) generated from the CW laser 706 on the transformed silk sample to detect the created fluorescent pattern 702 on the detector 718 by means of the focusing mirror 710 placed in between the CW laser 706 and the translation stage on which the transformed silk sample is placed.

In one embodiment, the focusing mirror 710 is placed in between the CW laser 706 and the translation stage in such a manner that the CW laser 706 is in a vertical direction with respect to the focusing mirror 710 and the translation stage is in a horizontal direction with respect to the focusing mirror 710.

In one embodiment, the CW wave laser 706 may have a wavelength of 532 nm corresponding to green excitation in order to detect the created fluorescent pattern 702 with the visible range of electromagnetic spectrum.

At block 808, the method 800 may include scanning through the microwave frequencies generated by the microwave resonator 704 to detect a magnetic resonance of the created fluorescent pattern at the one or more microwave frequencies by observing a change in the fluorescence intensity (In) at different microwave frequencies.

At block 810, the method 800 may include determining if a magnetic resonance is detected at one or more microwave frequencies. If a magnetic resonance is detected, the method 800 proceeds to block 814 and the method 800 stops as the occurrence of magnetic resonance confirms the electron-spin dependence of the created fluorescent pattern. However, if a magnetic resonance is not detected, the method 800 proceeds to block 812.

In one embodiment, the one or more frequencies at which magnetic resonance is detected is 1.44 GHz and 2.88 GHz.

At block 812, the method 800 may include moving the translation stage in at least one of the X, Y and Z directions to locate a fluorescent pattern on the transformed silk sample, different from the one detected at block 806. Once, the different fluorescent pattern is located on the transformed silk sample, the method 800, then proceeds to block 804 and follows the sequence of steps following block 804, until a magnetic resonance is detected.

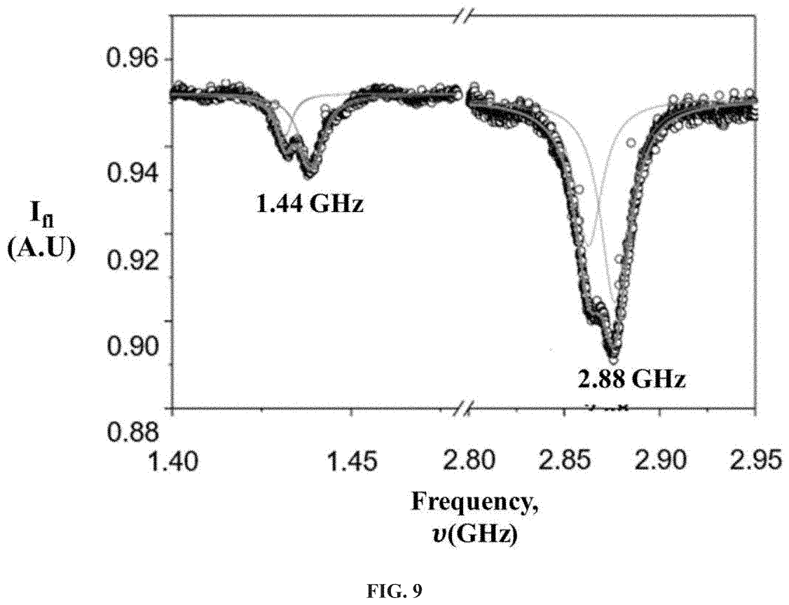

FIG. 9 depicts a plot of fluorescence intensity (I_{η}) versus microwave frequency at room temperature in accordance with an embodiment of the present disclosure. As seen from FIG. 9, the fluorescence intensity (I_{η}) of the detected fluorescent pattern on the transformed silk sample shows a sharp dip at 1.44 GHz and 2.88 GHz microwave frequencies. The dip in the fluorescence intensity (b) of the detected fluorescent pattern at the two microwave frequencies confirms magnetic resonance at 1.44 GHz and 2.88 GHz, thereby confirming the electron-spin dependence of the create fluorescent pattern.

To understand the mechanism behind the creation of electron-spin dependent fluorescence in silk, the transformed silk sample is analyzed using various structural and optical characterizations as described in the upcoming paragraphs.

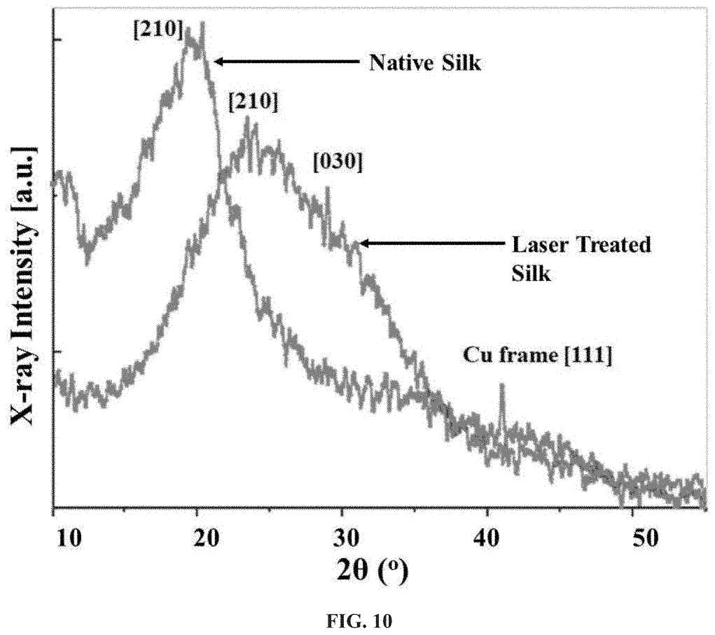

FIG. 10 depicts the XRD patterns of transformed silk and pristine silk in accordance with an embodiment of the present disclosure. The XRD patterns as depicted in FIG. 10 shows an atomic-scale compression in the nano-crystalline domains of the fluorescent silk when compared to the one in the native silk as also tabulated in table S1. The [210] plane in the control at 2θ=20°, shifts to larger angle around 23° and a new peak [030] is observed at 29°. This indicates about 11±3% reduction in mean lattice periodicity from 4.5 Å to near 4.0 Å. In addition, broadening in Bragg-peaks suggests an increase in inhomogeneity in the size of mean crystallites.

TABLE S1
XRD Analysis of Pristine and Transformed Silk

| Native Silk | | | Transformed Silk | | | Com- |
|---|---|---|---|---|---|---|
| | | Lattice | | | Lattice | pression |
| 2θ ± | Miller | Spacing, | 2θ ± | Miller | Spacing, | (D − Dₒ)/ |
| θ (°) | Indices | Dₒ (Å) | θ (°) | Indices | Dₒ (Å) | Dₒ |
| 10.5 ± | 010 | 8.5 | — | — | — | — |
| 0.8 | | | | | | |
| 17.3 ± | 020 | 5.2 | 18.9 ± | 020 | 4.8 | 7.5 |
| 1.5 | | | 2.2 | | | |
| 20.1 ± | 210 | 4.5 | 23.0 ± | 210 | 4.0 | 11.1 |
| 1.0 | | | 2.5 | | | |
| — | 210 | 4.5 | 29.1 ± | 030 | 3.2 | 28.9 |
| | | | 4.0 | | | |

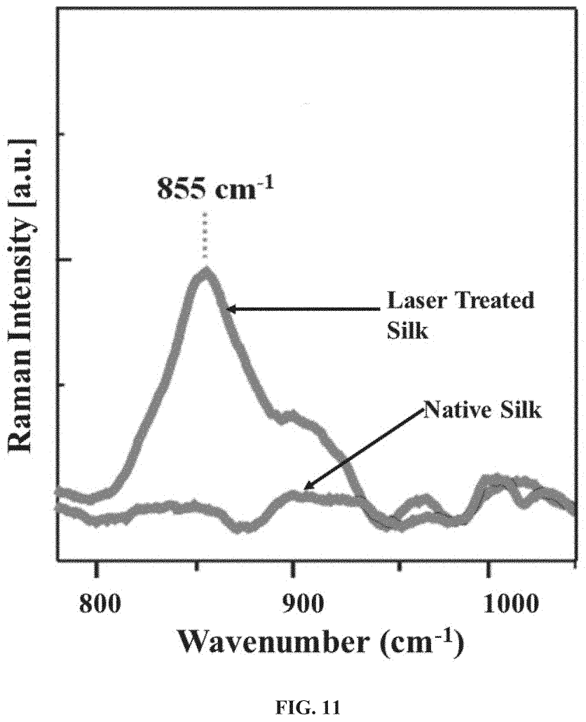

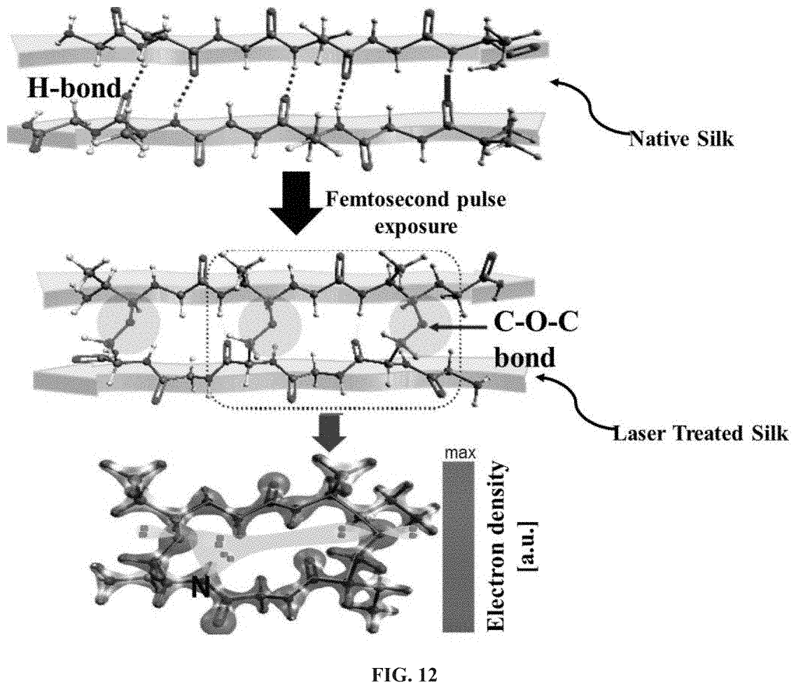

FIG. 11 depicts the Raman spectra of transformed silk and pristine silk in accordance with an embodiment of the present disclosure. The micro-Raman spectra of transformed silk is performed under 785 nm excitation to minimize the fluorescence background. The appearance of a new Raman band in transformed silk is observed at 855 cm⁻¹ which can be attributed to C—O—C bond vibration, while the other Raman bands remain preserved. This indicates that the femtosecond pulses disrupt the hydrogen bonding of the polar carboxyl group while perturbing other bonds in silk such as C—N and C—C as also shown in FIG. 12. Further, FIG. 12 also depicts trapping of electron lone-pairs in the crown ring structure, suggesting the emergence of electron spin-dependent fluorescence from mid-gap states in silk.

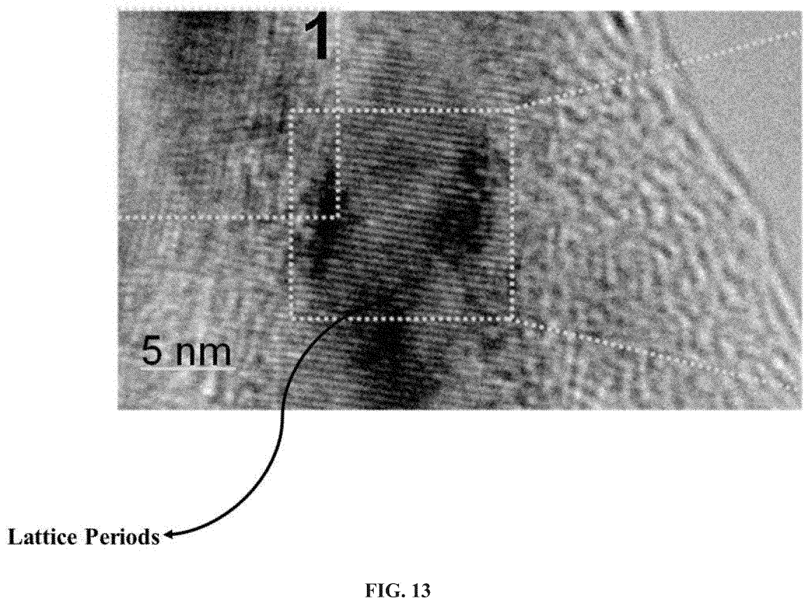

FIG. 13 depicts the HR-TEM image of the transformed silk in accordance with an embodiment of the present disclosure. The HR-TEM imaging of transformed silk is carried out by preparing silk nano-films on TEM grid having sub-200 nm thickness in order to prevent distortion of the transmitted electron-beam. The HR-TEM images shows the crystalline domains embedded in the amorphous background. The lattice periods of the fluorescent silk in the TEM images are in agreement with the ones obtained with XRD analysis, as seen in table S2, thereby validating that the femtosecond-pulses induce a compression in the nano-crystals.

TABLE S2
Comparison of lattice period in
nanocrystal from XRD and HR-TEM

| | XRD Analysis - Lattice | TEM Imagine - Lattice |
|---|---|---|
| 2θ (°) | Spacing (Å) | Spacing (Å) |
| 23.0 [210] | 4.0 | 3.7 ± 0.5 |
| 29.1 [030] | 3.2 | 3.0 ± 0.5 |

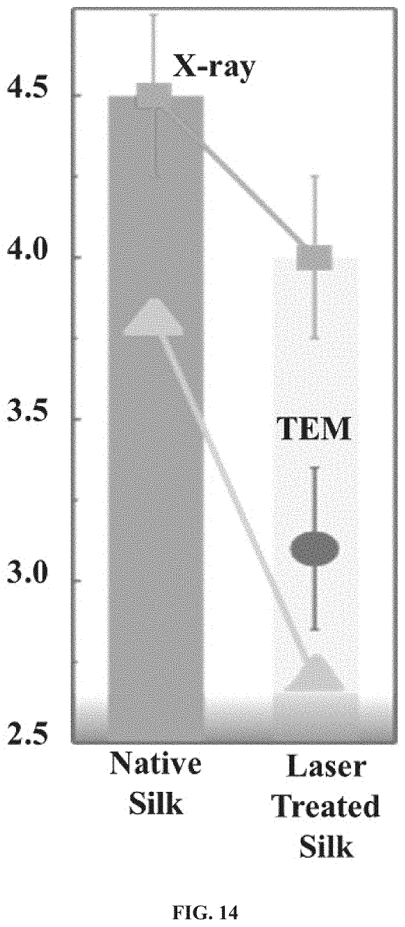

The compression of nano-crystals from pristine silk to transformed silk due to irradiation with femtosecond pulses and validated by XRD and HR-TEM imaging is also depicted in FIG. 14 that shows a variation in the lattice periodicity, d, for native and transformed silk and confirms a decrease in lattice periodicity when native silk is transformed upon irradiation with femtosecond pulses.

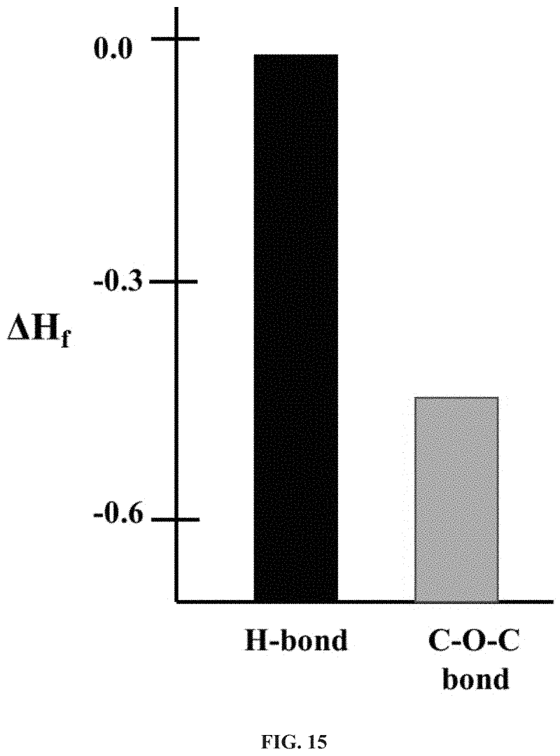

The transformation of hydrogen bonded strands of pristine silk on exposure to femtosecond pulses to C—O—C bonds is explained further by examining the enthalpy of formation (ΔH_{f}) of the hydrogen bonded strands and the C—O—C bonded strands and is depicted in FIG. 15. FIG. 15 shows that the enthalpy of formation for the C—O—C bonded strands is much lower than the hydrogen bonded strands indicating high stability of the C—O—C bond.

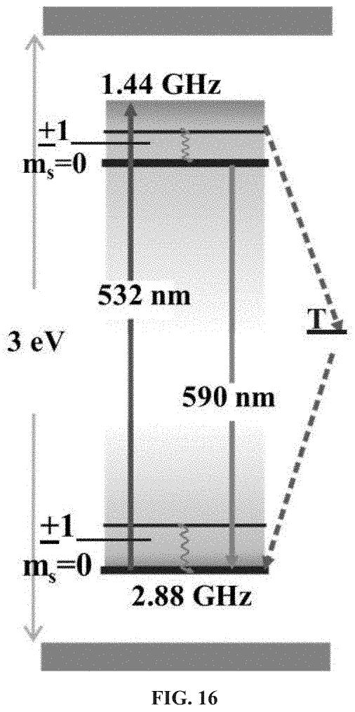

FIG. 16 depicts an experimental energy level diagram of spin-dependent fluorescent (SDF) silk in accordance with an embodiment of the present disclosure. As can be seen from FIG. 16, two singlet states separated by at least one triplet states in the 3 eV band gap are obtained. This confirms that optical excitation is spin-conserved, while the microwave magnetic field manipulates the fluorescence intensity at 2.88 GHz and 1.44 GHz resonances by controlling non-radiative decay by inter-system crossing (as shown by dashed lined in FIG. 16), thus explaining the essential mechanism of SDF.

The electron spin dependence fluorescence in transformed silk can be employed in various applications including but not limited to quantum thermometry, spin dependent fluorescence based imaging, magnetometry, single photon-source and quantum computing. Few of the applications are described in the upcoming paragraphs.

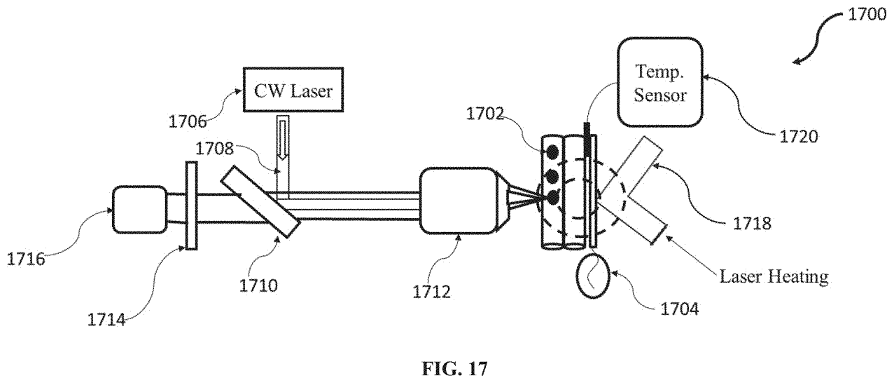

FIG. 17 depicts an experimental setup 1700 (herein after referred as “setup 1700”) for calibrating a fluorescent silk-based quantum thermometer in accordance with an embodiment of the present disclosure. The setup 1700 comprises a translation stage capable of moving along the X, Y and Z directions. The transformed silk sample is placed upon the translation stage. The setup 1700 further comprises a continuous wave (CW) laser 1706 placed diagonally to the translation stage and configured to generate a CW laser beam 1708 at a particular excitation wavelength within the visible range of the electromagnetic spectrum. The CW laser beam 1708 is directed towards the transformed silk sample by means of a focusing mirror 1710 so as to detect the created fluorescent pattern 1702 on the transformed silk sample. The setup 1700 further comprises a microwave resonator 1704 placed in close proximity to the transformed silk sample and capable of generating microwave frequencies within a range 1.4-3.0 GHz. The setup 1700 further comprises a second laser 1718 capable of heating the transformed silk sample. The setup 1700 further comprises a temperature sensor 1720 to detect the change in temperature when the transformed silk sample is heated by the second laser 1718. The setup 700 further comprises a filter 1714 to avoid excitation wavelength in fluorescence detection on the transformed silk sample. The setup further comprises a detector 1716 to detect a change in the fluorescence intensity with microwave frequencies. In one embodiment, the focusing mirror 710 and may be a dichroic mirror. In one embodiment, the filter 1714 may be a notch filter. In one embodiment, the detector 1716 may be an avalanche photo diode (APD). However, it may be understood may a skilled person that different combinations of the focusing mirror 1710, filter 1714 and detector 1716 may be used.

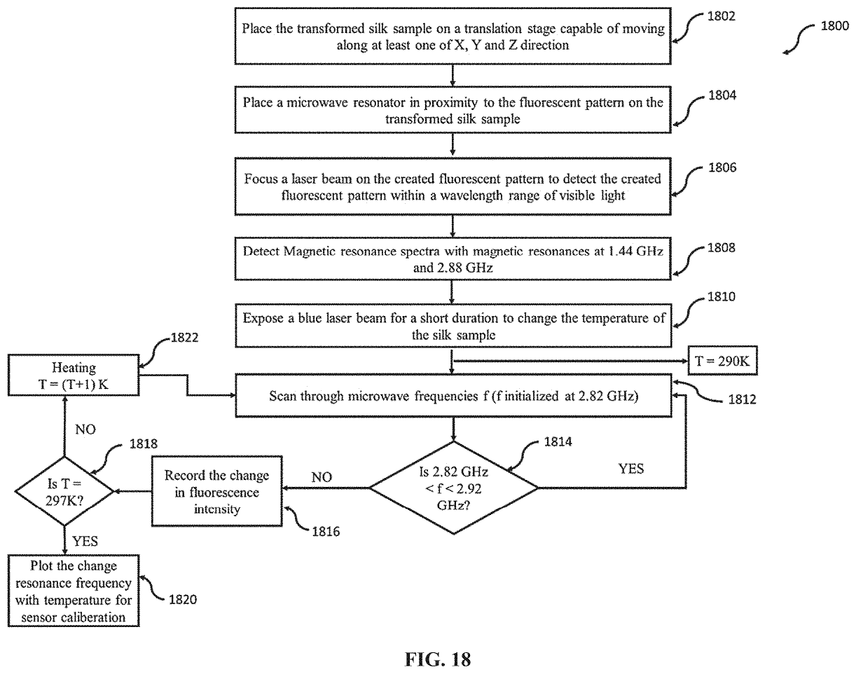

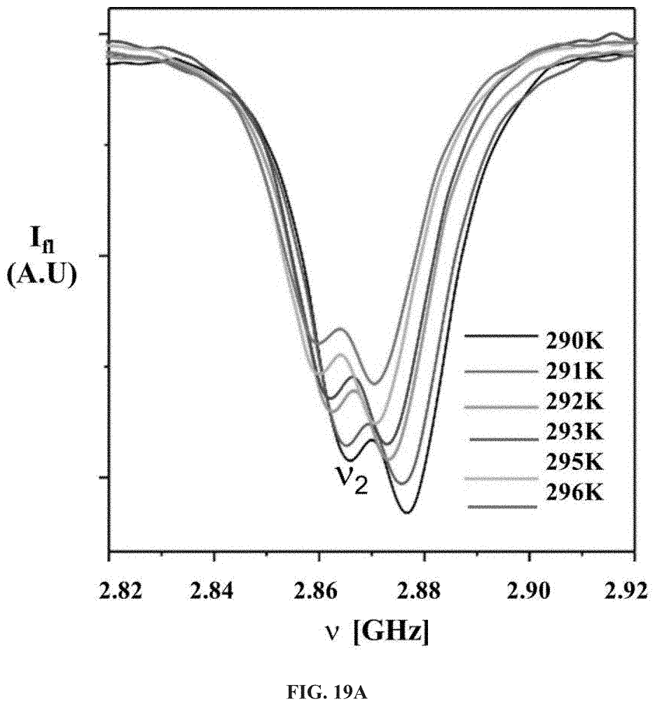

FIG. 18 depicts a method 1800 for calibrating fluorescent silk-based quantum thermometer in accordance with an embodiment of the present disclosure.

The order in which the method 1800 is described is not intended to be construed as a limitation, and any number of the described method blocks can be combined in any order to implement the method. Additionally, individual blocks may be deleted from the methods without departing from the spirit and scope of the subject matter described herein.

At block 1802, the method 1800 may include placing the transformed silk sample on the translation stage that is capable of moving in the X, Y and Z direction in order to detect the created fluorescent pattern 1702 on the transformed silk sample.

At block 1804, the method 1800 may include placing a microwave resonator 1704 in close proximity to the transformed silk sample and capable of generating microwave frequencies within a range 1.4-3.0 GHz. In one embodiment, the distance at which the microwave resonator 1704 is placed with respect to the transformed silk sample is 100-200 μm.

At block 1806, the method 1800 may include focusing a CW laser beam 1708 at one or more excitation powers (≤1 mW) generated from the CW laser 1706 on the transformed silk sample to detect the created fluorescent pattern 1702 on the detector 1716 by means of the focusing mirror 1710 placed in between the CW laser 1706 and the translation stage on which the transformed silk sample is placed.

In one embodiment, the focusing mirror 1710 is placed in between the CW laser 1706 and the translation stage in such a manner that the CW laser 1706 is in a vertical direction with respect to the focusing mirror 1710 and the translation stage is in a horizontal direction with respect to the focusing mirror 1710.

In one embodiment, the CW wave laser 1706 may have a wavelength of 532 nm corresponding to green excitation in order to detect the created fluorescent pattern 1702 with the visible range of electromagnetic spectrum.

At block 1808, the method 1800 may include scanning through the microwave frequencies generated by the microwave resonator 1704 to detect a magnetic resonance of the created fluorescent pattern at the one or more microwave frequencies by observing a change in the fluorescence intensity (In) at different microwave frequencies. In one embodiment, the one or more frequencies at which magnetic resonance is detected is 1.44 GHz and 2.88 GHz.

At block 1810, the method 1800 may include exposing a laser beam generated from the second laser 1718 on the transformed silk sample for a short duration in order to change the temperature of the transformed silk sample. Once, the temperature of the transformed silk sample as detected by the temperature sensor 1720 is 290K, the method 1800 proceeds to block 1812.

At block 1812, the method 1800 may include scanning the heated transformed silk sample through a range of microwave frequencies, f, starting from 2.82 GHz and up to 2.92 GHz.

At block 1814, the method 1800 may include detecting whether f lies between 2.82 GHz and 2.92 GHz. If the result of the detection is YES, the method 1800 falls back to block 1812. However, if the result of the detection is NO, the method 1800 proceeds to block 1816.

At block 1816, the method 1800 may include recording a change in the fluorescence intensity (I_{η}) with the change in temperature of the transformed silk sample.

At block 1818, the method 1800 may include detecting by the temperature sensor 1720 whether the temperature of the transformed silk has reached 297 K. If the result of the detection is YES, the method 1800 proceeds to block 1820. However, if the result of the detection is NO, the method 1800 proceeds to block 1822 and follows the sequence of steps starting from block 1812.

At block 1820, the method 1800 may include plotting the change in resonant frequency of the transformed and heated silk sample with temperature for sensor calibration, once the temperature of the transformed silk sample reaches 297 K.

At block 1822, the method 1800 may include heating the transformed silk sample further in order to raise its temperature by employing the laser beam generated from the second laser 1718 in increments of 1 K and thereafter following the sequence of steps starting from block 1812.

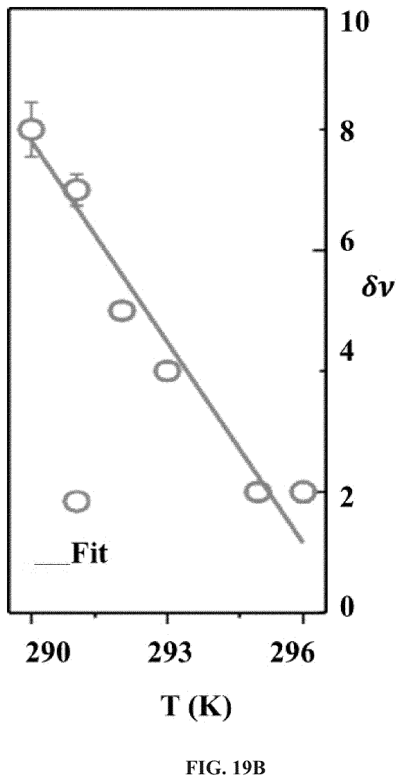

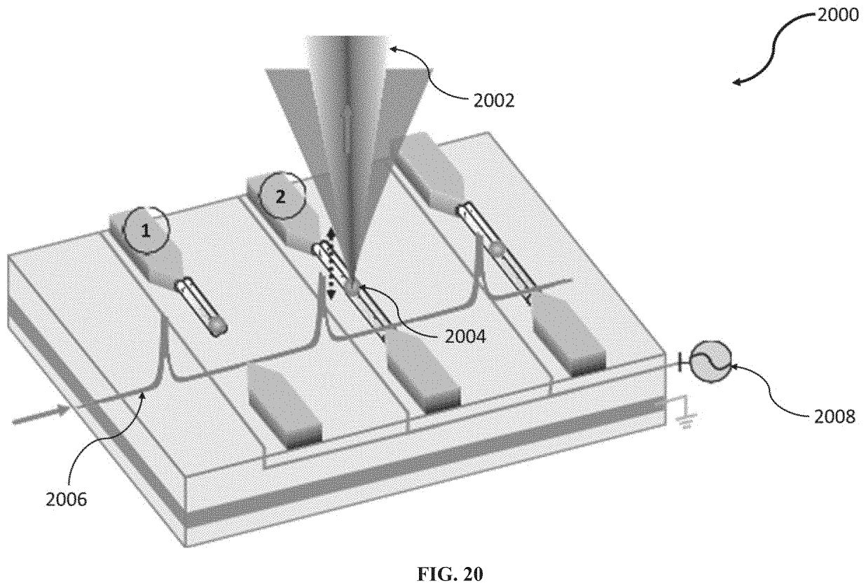

FIG. 19A depicts a shift in magnetic resonance induced by laser heating in accordance with an embodiment of the present disclosure. FIG. 19A depicts a shift in the 2.86 GHz resonance with increase in temperature from 290 K to 297 K. The shift in the resonance frequency with temperature is attributed as δν/δT=1 MHz/K as also can be seen from FIG. 19B that depicts a shift/change in frequency (δν) as a function of temperature, thereby rendering sub-K precision in ambient conditions, which is comparable to the ones based on color centers. This property of shifting the magnetic resonance with change in the temperature at sub-K precision for a given fluorescent pattern created on the silk sample reveals the possibility of calibrating and using of the fluorescent silk as a temperature sensor for temperature sensing.

FIG. 20 depicts a micro-chip 2000 with nano-mechanical silk-cantilever (device 1) and string-resonators (device-2) in accordance with an embodiment of the present disclosure. The microchip comprises a PCB chip on which the transformed silk sample is placed. The device marked 1 act as a cantilever and the device marked 2 acts as a string resonator. The micro-chip 2000 further comprises a CW laser capable of generating a CW laser beam 2002 on the transformed silk sample to detect the fluorescent pattern 2004. The micro-chip 2000 further comprises a microwave resonator capable of generating microwave frequencies in the range 1.4-3.0 GHz. The micro-chip 2000 further comprises means for transmitting an external acoustic pulse 2006 near the transformed silk sample for calibration.

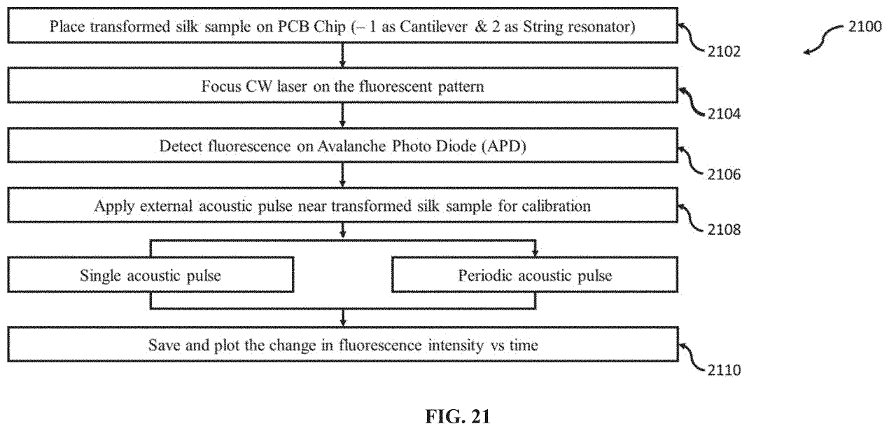

FIG. 21 depicts a method 2100 for employing transformed silk as nano-mechanical silk-cantilever (device 1) and string-resonators (device-2) in accordance with an embodiment of the present disclosure.

The order in which the method 2100 is described is not intended to be construed as a limitation, and any number of the described method blocks can be combined in any order to implement the method. Additionally, individual blocks may be deleted from the methods without departing from the spirit and scope of the subject matter described herein.

At block 2102, the method 2100 may include placing the transformed silk sample on the PCB chip.

At block 2104, the method 2100 may include focusing a continuous wave (CW) laser beam in order to detect a fluorescent pattern on the transformed silk sample.

At block 2106, the method 2100 may include detecting the created fluorescent patter on a detector. In one embodiment, the detector may be an avalanche photo diode (APD). However, it may be understood that any other suitable detector may also be used for detecting the fluorescent pattern on the transformed silk sample.

At block 2108, the method 2100 may include applying external acoustic pulse near transformed silk sample for calibration of devices 1 and 2. In one embodiment, the external acoustic pulse may be a single acoustic pulse. In another embodiment, the external acoustic pulse may be a periodic acoustic pulse.

At block 2110, the method 2100 may include plotting a change in fluorescence intensity of the transformed silk sample with time.

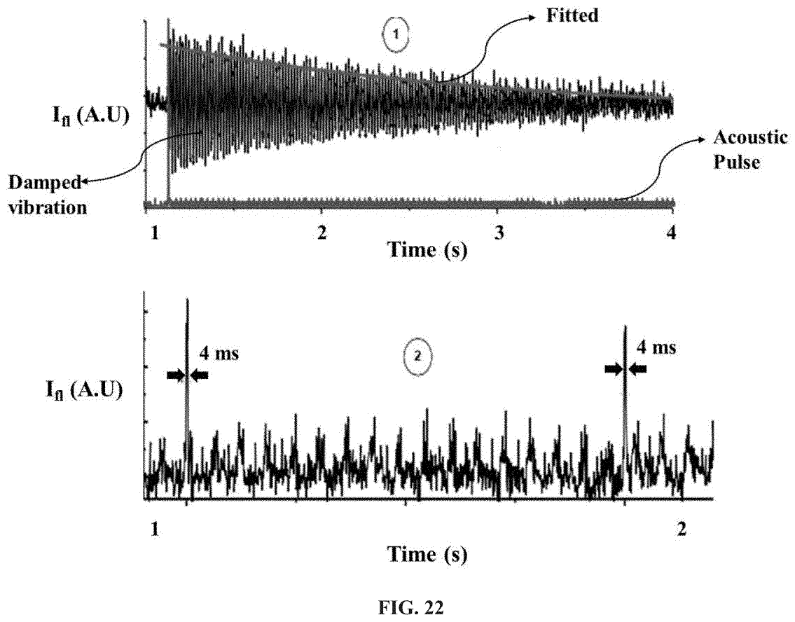

FIG. 22 depicts optical detection of mechanical response by acoustic excitation of devices 1 and 2 in accordance with an embodiment of the present disclosure. For device 1, the damped oscillations of the transformed silk-based cantilever is measured with a quality factor, Q, of almost equal to 300 in air. For device 2, the impulse response of the silk-string is measured. The fluorescence in silk enabled an optical readout to measure real-time position of micro-fiber subjected to, for example, acoustic excitation, which is further calibrated and used as an acoustic wave/vibration sensor on the basis of its configuration. Exhibiting such property in the fluorescent silk reveals a fact that fluorescent silk may be calibrated and used as various acoustic wave/vibration sensor based on its configuration.

A description of an embodiment with several components in conjunction with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system for creating electron spin dependent fluorescence at nano scale level in a protein, the system comprising:
a translation stage with the protein placed thereupon, wherein the translation stage is capable of moving along at least one of X, Y and Z direction;
a femtosecond pulse laser placed diagonally to the translation stage and configured to generate a femtosecond pulse to create a fluorescence pattern at nanoscale level on the protein by moving the translation stage along the at least one of X, Y and Z direction, wherein the generated femtosecond pulse is directed towards the protein sample by a first focusing mirror placed between the femtosecond pulse laser and the translation stage in such a manner that the femtosecond laser is in a vertical direction with respect to the first focusing mirror and the translation stage in a horizontal direction with respect to the first focusing mirror, wherein the fluorescence pattern at nanoscale is created by irradiating the protein with said femtosecond pulse at one or more irradiation fluences in a room temperature in air;
a continuous wave laser placed diagonally to the translation stage and configured to generate a laser beam to detect the created fluorescence pattern on the protein sample within a wavelength range of visible light, wherein the generated laser beam is directed towards the protein sample by a second focusing mirror placed between the continuous wave laser and the translation stage in such a manner that the continuous wave laser is in a vertical direction with respect to the second focusing mirror and the translation stage in a horizontal direction with respect to the second focusing mirror;
a microwave resonator capable of providing one or more frequencies and placed in close proximity to the protein on which the fluorescence pattern is created; and
a detector placed axially from the translation stage, configured to detect a magnetic resonance of the created fluorescence pattern at least at one of the one or more frequencies.

2. The system of claim 1, wherein the protein is spider silk (Araneous neoscona) or silkworm silk (Bombymx mori).

3. The system of claim 1, wherein:
the femtosecond laser has a wavelength of 700-800 nm and having an energy of 2 nJ-2 mJ/pulse,
a pulse width of the femtosecond pulse lies within a range of 7-25 femtoseconds, and
the one or more irradiation fluence values lies within a range of 1-1000 mJ/cm².

4. The system of claim 1, wherein:
the microwave resonator is placed at a distance ranging between 100-200 μm from the created fluorescence pattern,
the one or more frequencies provided by the microwave resonator lies within a range of 1.4-3.0 GHz, and
the at least one of the one or more frequencies at which magnetic resonance is detected comprises at least one of 1.44 GHz and 2.88 GHz.

5. The system of claim 1, wherein the detector is an avalanche photo diode or a single photon detector, and the magnetic resonance is detected by observing a change in an intensity of the created fluorescence pattern at the at least one of the one or more frequencies.

6. The system of claim 1, wherein the fluorescence pattern at nanoscale level is created in a selected environment and fixed to air, vacuum, or argon.

7. The system of claim 1, wherein the created fluorescence pattern comprises a fluorescent nano-dot or a fluorescent line or any other arbitrary shape, wherein the created fluorescence pattern has a width having a range 100 to 250 nm.

8. The system of claim 1, wherein translation stage is moved along at least one of X, Y and Z direction to create multiple dots by raster scanning the protein.

9. A system for creating electron spin dependent bulk fluorescence in a protein, the system comprising:
a translation stage with the protein placed thereupon, wherein the translation stage is capable of moving along at least one of X, Y and Z direction;
a continuous wave laser placed diagonally to the translation stage and configured to generate a laser beam to create and detect the created fluorescence pattern on the protein sample within a wavelength range of visible light, wherein the generated laser beam is directed towards the protein sample by a focusing mirror placed between the continuous wave laser and the translation stage in such a manner that the continuous wave laser is in a vertical direction with respect to the focusing mirror and the translation stage in a horizontal direction with respect to the focusing mirror, wherein the fluorescence pattern is created and detected by irradiating the protein using the laser beam at room temperature and at predetermined pressure ranging from 0.1 to 10 Gpa;
a microwave resonator capable of providing one or more frequencies and placed in proximity to the protein on which the fluorescence pattern is created; and
a detector placed axially from the translation stage, configured to detect a magnetic resonance of the created fluorescence pattern at least one of the one or more frequencies.

10. A method for creation of electron spin dependent fluorescence at nano scale level in a protein, the method comprising:
i. creating a fluorescence pattern at nano scale level on the protein by irradiating the protein using femtosecond pulses generated from a femtosecond pulse laser at one or more irradiation fluences at room temperature, wherein a value of the one or more irradiation fluences is less than a value of an ablation threshold of the protein;
ii. detecting the created fluorescence pattern by directing a laser beam towards the transformed protein, wherein the laser beam is generated by a continuous wave laser having a wavelength within a wavelength range of visible light;
iii. providing a microwave resonator, placed in proximity to the protein on which fluorescence pattern is created, wherein the microwave resonator is capable of providing a plurality of frequencies; and
iv. confirming an electron spin dependent fluorescence pattern by detecting, at a detector, a magnetic resonance of the created fluorescence pattern for at least one of the plurality of frequencies.

11. The method of claim 10, wherein the fluorescence pattern at nanoscale level is created in at least one of air, vacuum, and argon environment.

12. The method of claim 10, wherein the protein is spider silk (Araneous neoscona) or silkworm silk (Bombymx mori).

13. The method of claim 10, wherein the protein is placed on a translation stage capable of moving along at least one of X, Y and Z direction in order to create the fluorescence pattern with a precision of at least 100 nm.

14. The method of claim 10, wherein creating the fluorescence pattern comprises:
transforming the molecular structure of the protein by inducing structural modifications in the protein at the nano scale level by forming a stable C—O—C bonded crown-ring structure with localized electrons;
generating mid-gap states within a bandgap of silk spectra having life-time in a range of 10-50 ns of the molecular structure; and
creating, a spin dependent mid-gap fluorescence in a visible range at room temperature by exiting and emitting radiation within the mid gap states.

15. The method of claim 10, wherein the method involves laser-induced compression in β-sheets of protein resulting in formation of stable C—O—C bonded crown-ring structure with localized electrons leading to mid-gap states having fluorescence.

16. The method of claim 10, wherein the created fluorescence pattern comprises a fluorescent nano-dot or a fluorescent line or other arbitrary shape, wherein the created fluorescence pattern has a width having a range 100 to 250 nm; and wherein the method further comprises raster scanning the protein to create multiple dots.

17. A method for creation of electron spin dependent bulk fluorescence in a protein, the method comprising:
(i) creating and detecting a bulk fluorescence pattern on the protein by irradiating the protein using a laser beam at room temperature in a selected environment and fixed to air, vacuum, or argron wherein the laser beam is generated by a continuous wave laser having a wavelength within a wavelength range of visible light;
(ii) providing a microwave resonator, placed in close proximity to the protein on which fluorescence pattern is created, wherein the microwave resonator is capable of providing a plurality of frequencies; and
(iii) confirming an electron spin dependent fluorescence pattern by detecting, at a detector, a magnetic resonance of the created fluorescence pattern for at least one of the plurality of frequencies.

18. The method of claim 17, wherein the bulk fluorescence pattern is created in a selected environment and fixed to air, vacuum, or argon.

19. A transformed protein having altered molecular structure, obtained by the system of claim 1, wherein the protein exhibits modified quantum properties and fluorescence that is nano-confined, dopant-free, electron spin-dependent, or a combination thereof.

20. The transformed protein of claim 19, wherein:
the protein has potential applications in reconfigurable quantum sensing, spin dependent fluorescence-based imaging, quantum computing, a single photon-source, or a combination thereof;
the protein is utilized in an all-optical thermometer that demonstrates solid-state quantum sensing of local temperature by measuring thermal-induced reversible shift in the spin-resonance; and
the protein is utilized in hybrid nano-mechanical ultrasensitive cantilevers on a chip and achieve real-time acoustic sensing.
